(19) United States
(12) Patent Application Publication
(10) Pub. No.: US 2024/0350458 A1
(43) Pub. Date: Oct. 24, 2024

(54) 14-3-3 PROTEIN MODULATORS AS ANTITUMOR AGENTS

(71) Applicants: Simona Rapposelli, Capannori (LU) (IT); Eugenio Gaudio, Bellinzona (CH); Fabrizio Dal Piaz, Napoli (IT)

(72) Inventors: Simona Rapposelli, Capannori (LU) (IT); Eugenio Gaudio, Bellinzona (CH); Fabrizio Dal Piaz, Napoli (IT)

(21) Appl. No.: 18/580,822

(22) PCT Filed: Jul. 21, 2022
(86) PCT No.: PCT/EP2022/070443

§ 371 Date: Jan. 19, 2024

(30) Foreign Application Priority Data
Jul. 22, 2021 (IT) 102021000019526

(51) Int. Cl. A61K 31/4178; A61K 31/404; A61K 31/4155; A61K 31/4439; A61P 5/00; C07D 209/34; C07D 401/06; C07D 403/06; C07D 409/06

(52) U.S. Cl. CPC A61K 31/4178; A61K 31/404; A61K 31/4155; A61K 31/4439; A61P 5/00; C07D 209/34; C07D 401/06; C07D 403/06; C07D 409/06

(57) ABSTRACT

The invention relates to a 2-oxoindole compound of Formula (I) or a pharmaceutically acceptable salt thereof, wherein R₁ and R₂ are, independently from each other, hydrogen, 1H-imidazolyl, thienyl and 1-methyl-1H-imidazolyl, 2-methyl-1H-imidazolyl, 2-aminopyridinyl, 1H-pyrazolyl, with the proviso that one of R₁ and R₂ is hydrogen; or R₁ and R₂ together form 9H-fluorene R₃ is hydrogen, (C₁-C₃)alkyl, halogen or NO₂; for use as a 14-3-3 protein modulator of a tumor selected from the group consisting of a lymphoma, chronic lymphocytic leukaemia (CLL), Ewing sarcoma, colon cancer, melanoma and anaplastic thyroid cancer (ATC). The invention relates also new 2-oxoindole compound of Formula (I) or a pharmaceutically acceptable salt thereof.

### FIELD OF THE INVENTION

The present invention relates to 14-3-3 protein modulators as antitumor agents.

### BACKGROUND

14-3-3 proteins are a family of highly conserved cellular proteins that play key roles in the regulation of central pathways, both in physiological conditions and in diseases, such as cancer and neurodegenerative disorders. Several 14-3-3 target proteins (>200) have been identified, including proteins involved in mitogenic signalling, cell survival, cell cycle control, apoptosis, transcriptional regulation, cellular metabolism and cytoskeletal integrity [Tzivion G, Gupta V S, Kaplun L, Balan V: 14-3-3 proteins as potential oncogenes. Seminars in cancer biology 2006, 16:203-213]. Importantly, the involvement of 14-3-3 proteins in the regulation of various oncogenes and tumour suppressor genes support its detrimental role in human cancer [Wilker E, Yaffe M B: 14-3-3 Proteins—a focus on cancer and human disease. Journal of molecular and cellular cardiology 2004, 37:633-642]. In humans, seven genes codify for seven 14-3-3 proteins, namely alpha/beta, epsilon, eta, gamma, theta, sigma, and zeta/delta. Their expression is common to all human tissues, while the overexpression is mostly associated with cancer's insurgence and correlated with advanced tumour grade. 14-3-3 proteins are known to interact with Beclin-1 and by inhibiting autophagy, they promote the tumorigenesis in lung cancer [Kidd M E, Shumaker D K, Ridge K M: The role of vimentin intermediate filaments in the progression of lung cancer. American journal of respiratory cell and molecular biology 2014, 50:1-6], glioma, renal cell carcinoma, and cervical cancer. Under certain conditions, the same proteins could activate the autophagic process in glioblastoma and pancreatic. Similarly, in neurodegenerative diseases, 14-3-3 proteins regulate autophagy, which promotes the degradation of accumulated protein [Martina J A, Diab H I, Lishu L, Jeong A L, Patange S, Raben N, Puertollano R: The nutrient-responsive transcription factor TFE3 promotes autophagy, lysosomal biogenesis, and clearance of cellular debris. Science signaling 2014, 7:ra9].

14-3-3 have been indicated as targets of therapy in 2011 [Zhao J, Meyerkord C L, Du Y, Khuri F R, Fu H: 14-3-3 proteins as potential therapeutic targets. Seminars in cell & developmental biology 2011, 22:705-712] and since then some campaigns of drug discovery took place but up today there are no agents in clinical development. By applying the phage display technology, the R18 peptide was identified to compete with client proteins for the binding to 14-3-3 proteins. R18 is available for research studies and represents the most advanced inhibitor of 14-3-3 proteins. The object of the present invention is hence to provide 14-3-3 protein modulators in order to find molecules capable to compete on the binding to 14-3-3 proteins.

The inventors principally focused their attention on 2-oxoindole derivates, wherein one of the inventors proposed oxoindole derivates and described them as agents able to affect Akt pathway [Sestito S, Nesi G, Daniele S, Martelli A, Digiacomo M, Borghini A, Pietra D, Calderone V, Lapucci A, Falasca M, et al: Design and synthesis of 2-oxindole based multi-targeted inhibitors of PDK1/Akt signaling pathway for the treatment of glioblastoma multiforme. European journal of medicinal chemistry 2015, 105:274-288].

In fact, Rapposelli S. et al synthesized small molecules to be used in combination with antitumor drug agents and described it in WO2016/055454 as agents capable to synergize the inhibition of PDK1/Akt signaling pathway. Specifically, N-[(3Z)-3-(1H-imidazol-5-ylmethylidene)-2-oxo-2,3-dihydro-1H-indol-5-yl]-4-methyl-benzenesulfonamide, when combined with temozolomide, induced a significant increase in inhibition the glioblastoma cell viability with respect to the single treatment with compound N-[(3Z)-3-(1H-imidazol-5-ylmethylidene)-2-oxo-2,3-dihydro-1H-indol-5-yl]-4-methyl-benzenesulfonamide (at all concentration tested) or with temozolomide (example 2 and FIG. 3).

Therefore, the patent publication WO2016/055454 referred to the use of a combination for the treatment of glioblastoma multiforme (GBM), breast tumor and pancreatic tumor together with antitumor agents.

### SUMMARY OF THE INVENTION

Even if the results of compound N-[(3Z)-3-(1H-imidazol-5-ylmethylidene)-2-oxo-2,3-dihydro-1H-indol-5-yl]-4-methyl-benzenesulfonamide, when used alone against glioblastoma cells, was not evidently promising, the inventors tried to test 2-oxoindole derivatives as 14-3-3 protein modulators.

As it will be clear below with the experimental part, the inventors surprisingly found out that only a small group of 2-oxoindole derivatives were capable to act against rare forms of cancer and lethal haematological cancers and soft tissue sarcoma as a new class of 14-3-3 protein modulators able to affect human cancer growth both in vitro and in vivo models.

Therefore, the present invention concerns a 2-oxoindole compound of Formula (I) or a pharmaceutically acceptable salt thereof

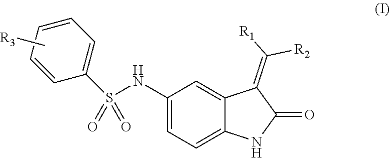

wherein
R₁ and R₂ are, independently from each other, hydrogen, 1H-imidazolyl, thienyl and 1-methyl-1H-imidazolyl, 2-methyl-1H-imidazolyl, 2-aminopyridinyl, 1H-pyrazolyl, with the proviso that one of R₁ and R₂ is hydrogen; or R1 and R2 together form 9H-fluorene
R₃ is hydrogen, (C₁-C₃)alkyl; halogen or NO₂
for use as a 14-3-3 protein modulator of a tumor selected from the group consisting of a lymphoma, chronic lymphocytic leukemia (CLL), Ewing sarcoma, colon cancer, melanoma and anaplastic thyroid cancer (ATC).

The inventors hence found out the compounds of formula (I) were optimal modulators of 14-3-3 proteins as it will be evident in the detailed description.

In another aspect the invention concerns a new family of compounds of Formula (II) Therefore, the present invention concerns also a 2-oxoindole compound of Formula (I) or a pharmaceutically acceptable salt thereof

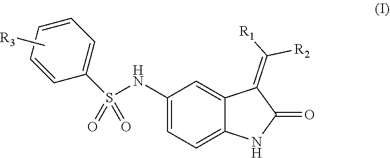

wherein
R₁ and R₂ are, independently from each other, hydrogen, thienyl and 2-methyl-1H-imidazolyl, 2-aminopyridinyl, 1H-pyrazolyl, with the proviso that one of R₁ and R₂ is hydrogen; or R1 and R2 together form 9H-fluorene
R₃ is hydrogen, (C₁-C₃)alkyl; halogen or NO₂,
with the proviso that when R₁ or R₂ is thienyl, then R₃ is not methyl.

In a further aspect the invention concerns a new 2-oxoindole compound or a pharmaceutically acceptable salt thereof for use as a medicament.

The new 2-oxoindole compound of Formula (I) or a pharmaceutically acceptable salt thereof can be used as a 14-3-3 protein modulator of a tumor. The tumor is preferably selected from the group consisting of a lymphoma, chronic lymphocytic leukemia (CLL), Ewing sarcoma, colon cancer, melanoma and anaplastic thyroid cancer (ATC).

### BRIEF DESCRIPTION OF THE DRAWINGS

FIG. 1 shows the results of Human Anaplastic thyroid cancer (hATC) cell lines incubated with FC86 for 72 h and then assayed with MTT as reported in the example 4;

FIG. 2 shows the results of tests wherein RNA extracted from thyroid cancer cell lines was evaluated by Real Time PCR for the baseline expression of 14-3-3 proteins as reported in the example 4;

FIG. 3 shows the results of tests as reported in example 5 wherein (A) FRTL5 cells, derived from normal rat thyroid and (B) healthy human thyroid cells were treated with a range concentration of FC86 for 72 h. FRTL5 were assayed with MTT, while healthy human thyroid cells by trypan blue staining. The percentage of dead cells, as counted after trypan blue staining, was around 10-15% at the dose of 800 nM of FC86.

FIG. 4 represents DARTS experiments results as reported in example 6: Proteins obtained from U937 cells incubated with 50 nM FC86 or with the medium for 2 h underwent subtilisin-catalyzed digestion. The resulting mixtures were analysed by MS-based (A) or western blot WB (B) revealing a significant increment of resistance to proteolysis of the protein 14-3-3 following the incubation with compound FC86;

FIG. 5 represents DARTS experiments results as reported in example 6: Proteins obtained from U87MG cells incubated with 5 μM of FC86 or with the medium for 2 h underwent subtilisin-catalyzed digestion. The resulting mixtures were analysed by MS-based (A) or WB (B) revealing a significant increment of resistance to proteolysis of the protein 14-3-3 following the incubation with FC86.

FIG. 6 reports ADME-TOX profiling of compounds FC91, FC85, and FC86 as explained in example 7 FIG. 7 reports the inhibition of the compound FC86 on tumor growth in xenograft models of lymphoma (TMD8 engrafted in NOD-Scid mice); vehicle group (blue line), n=9 mice; FC86 treated group (red line), n=9 mice (Example 8). Tumor volume expressed in cubic mm plus SEM (Student t-test); p<0.01.

FIG. 8 shows the results of tests of example 9 wherein (A) Peripheral blood mononuclear cells were isolated from healthy donors by ficoll extraction. FC86 was incubated with cells at the concentration of 200 nM for 48 h. Annexin V was detected by flow cytometry (FACS). PBMCs (Peripheral blood mononuclear cells) were not affected by FC86.

### DETAILED DESCRIPTION OF THE INVENTION

The present invention concerns a 2-oxoindole compound of Formula (I) or a pharmaceutically acceptable salt thereof

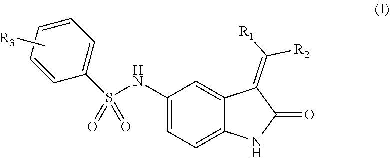

wherein
R₁ and R₂ are, independently from each other, hydrogen, 1H-imidazolyl, thienyl and 1-methyl-1H-imidazolyl, 2-methyl-1H-imidazolyl, 2-aminopyridinyl, 1H-pyrazolyl, with the proviso that one of R₁ and R₂ is hydrogen; or R₁ and R₂ together form 9H-fluorene
R₃ is hydrogen, (C₁-C₃)alkyl, halogen or NO₂;
for use as a 14-3-3 protein modulator of a tumor selected from the group consisting of a lymphoma, chronic lymphocytic leukaemia (CLL), Ewing sarcoma, colon cancer melanoma and anaplastic thyroid cancer (ATC).

R₁ and R₂ are, independently from each other, hydrogen, 1H-imidazolyl, thienyl and 1-methyl-1H-imidazolyl, 2-methyl-1H-imidazolyl, 2-aminopyridinyl, 1H-pyrazolyl with the proviso that one of R₁ and R₂ is not hydrogen or R1 and R2 together form 9H-fluorene.

In a preferred embodiment R₁ or R₂ is 1H-pyrazolyl, more preferably 1H-pyrazol-2-yl or 1H-pyrazol-5-yl.

In a preferred embodiment R₁ or R₂ is 1H-imidazolyl, more preferably 1H-imidazol-2-yl or 1H-imidazolyl-5-yl.

In a preferred embodiment one of R₁ and R₂ is thienyl.

R₃ is hydrogen, (C₁-C₃)alkyl, halogen or NO₂, more preferably R₃ can be methyl, ethyl, propyl, isopropyl, fluoro or NO₂, still more preferably it is methyl. In an advantageous form R₃ is a methyl in 4 position. When R₃ is halogen it is preferably fluoro in 4-position.

The compounds of the invention can be in form E and Z with reference to the definition of R₁ and R₂.

In a preferred embodiment the compound of formula (I) is selected from the group consisting of

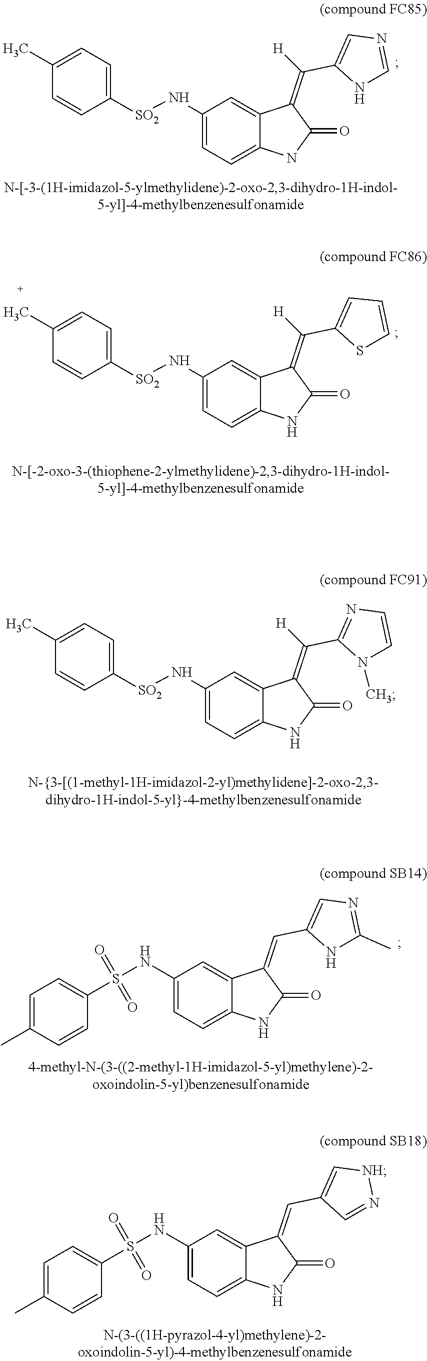

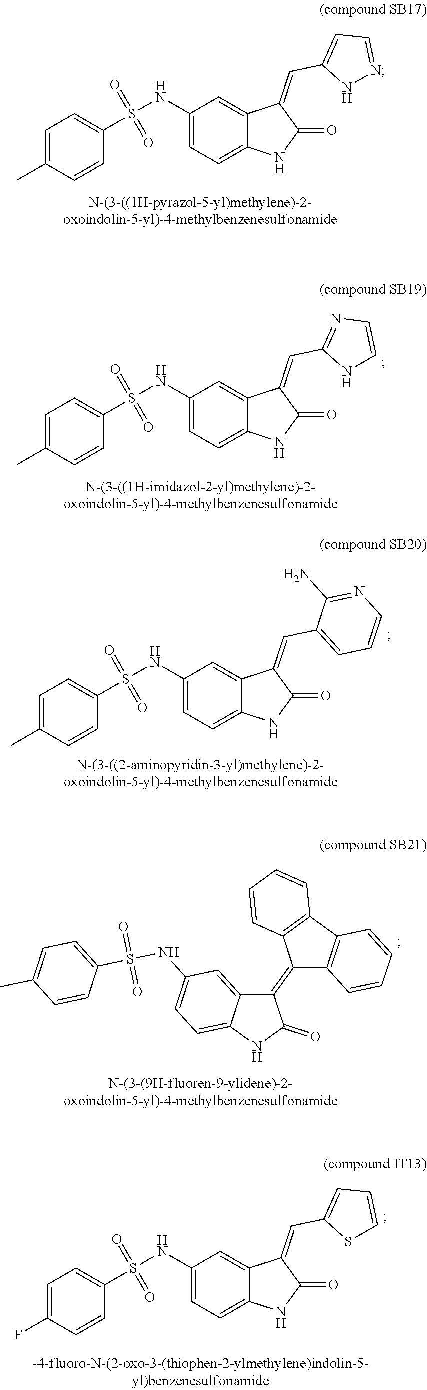 [SMILES: Cc1ccc(S(=O)(=O)Nc2ccc3c(c2)/C(=C/c2ccn[nH]2)C(=O)N3)cc1 ; Cc1ccc(S(=O)(=O)Nc2ccc3c(c2)/C(=C/c2ncc[nH]2)C(=O)N3)cc1 ; Cc1ccc(S(=O)(=O)Nc2ccc3c(c2)/C(=C/c2cccnc2N)C(=O)N3)cc1 ; Cc1ccc(S(=O)(=O)Nc2ccc3c(c2)C(=C2c4ccccc4-c4ccccc42)C(=O)N3)cc1 ; O=C1Nc2ccc(NS(=O)(=O)c3ccc(F)cc3)cc2/C1=C/c1cccs1]

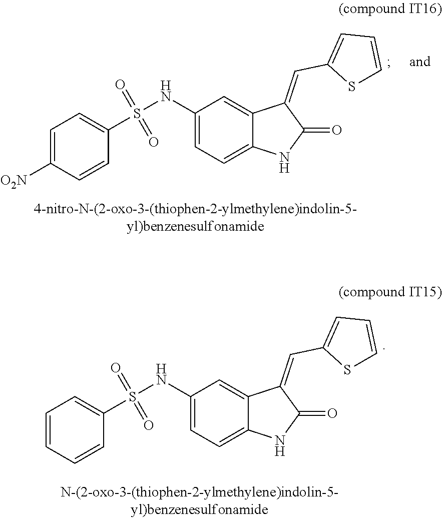 [SMILES: O=C1Nc2ccc(NS(=O)(=O)c3ccc([N+](=O)[O-])cc3)cc2/C1=C/c1cccs1 ; O=C1Nc2ccc(NS(=O)(=O)c3ccccc3)cc2/C1=C/c1cccs1]

In case of FC86 it can be in form E, i.e. N-[(3E)-2-oxo-3-(thiophene-2-ylmethylidene)-2,3-dihydro-1H-indol-5-yl]-4-methylbenzenesulfonamide) or in form Z, i.e. (N-[(3Z)-2-oxo-3-(thiophene-2-ylmethylidene)-2,3-dihydro-1H-indol-5-yl]-4-methylbenzenesulfonamide).

According to the present invention, all the compounds can be in E and Z forms, being comprised in the definitions all the stereo-compounds.

Therefore in a most preferred embodiment the compound for use of Formula (I) is selected from the group consisting of:
N-[(3Z)-3-(1H-imidazol-5-ylmethylidene)-2-oxo-2,3-dihydro-1H-indol-5-yl]-4-methylbenzenesulfonamide (compound FC85);
N-[(3Z)-2-oxo-3-(thiophene-2-ylmethylidene)-2,3-dihydro-1H-indol-5-yl]-4-methylbenzenesulfonamide (compound FC86);
N-{(3E)-3-[(1-methyl-1H-imidazol-2-yl)methylidene]-2-oxo-2,3-dihydro-1H-indol-5-yl}-4-methylbenzenesulfonamide (compound FC91);
(Z)-4-methyl-N-(3-((2-methyl-1H-imidazol-5-yl)methylene)-2-oxoindolin-5-yl)benzenesulfonamide (compound SB14);
(E/Z)—N-(3-((1H-pyrazol-4-yl)methylene)-2-oxoindolin-5-yl)-4-methylbenzenesulfonamide (compound SB18);
(Z)—N-(3-((1H-pyrazol-5-yl)methylene)-2-oxoindolin-5-yl)-4-methylbenzenesulfonamide (compound SB17);
(Z)—N-(3-((1H-imidazol-2-yl)methylene)-2-oxoindolin-5-yl)-4-methylbenzenesulfonamide (compound SB19);
N-(3-((2-aminopyridin-3-yl)methylene)-2-oxoindolin-5-yl)-4-methylbenzenesulfonamide (compound SB20);
N-(3-(9H-fluoren-9-ylidene)-2-oxoindolin-5-yl)-4-methylbenzenesulfonamide (compound SB21);
(Z)-4-fluoro-N-(2-oxo-3-(thiophen-2-ylmethylene)indolin-5-yl)benzenesulfonamide (compound IT13);
(Z)-4-nitro-N-(2-oxo-3-(thiophen-2-ylmethylene)indolin-5-yl)benzenesulfonamide (compound IT16); and
(Z)—N-(2-oxo-3-(thiophen-2-ylmethylene)indolin-5-yl)benzenesulfonamide (compound IT15).

Preferably compound FC86 was extremely active against lymphomas, chronic lymphocytic leukemia (CLL), Ewing sarcoma, colon cancer, melanoma and anaplastic thyroid cancer (ATC).

The compounds of Formula (I) are antitumor agents against lymphoma, chronic lymphocytic leukemia (CLL), Ewing sarcoma, colon cancer, melanoma and anaplastic thyroid cancer (ATC). Preferably the compounds of the invention showed surprisingly results against lymphomas, melanoma and ATC.

The inventors found out some new compounds of formula (I) that were optimal modulators of 14-3-3 proteins as it will be evident in the detailed description.

In another aspect the invention hence concerns a new family of compounds of Formula (I) Therefore, the present invention concerns also a 2-oxoindole compound of Formula (I) or a pharmaceutically acceptable salt thereof

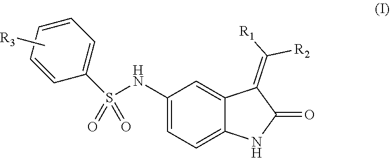

wherein
R₁ and R₂ are, independently from each other, hydrogen, 1H-imidazoly-2-yl, thienyl and 2-methyl-1H-imidazolyl, 2-aminopyridinyl, 1H-pyrazolyl, with the proviso that one of R₁ and R₂ is hydrogen; or R1 and R2 together form 9H-fluorene
R₃ is hydrogen, (C₁-C₃)alkyl; halogen or NO₂,
with the proviso that when R₁ or R₂ is thienyl, then R₃ is not methyl.

R₁ and R₂ are, independently from each other, hydrogen, 1H-imidazoly-2-yl, thienyl and 2-methyl-1H-imidazolyl, 2-aminopyridinyl, 1H-pyrazolyl, with the proviso that one of R₁ and R₂ is hydrogen; or R₁ and R₂ together form 9H-fluorene and with the proviso that when R₁ or R₂ is thienyl, then R₃ is not methyl.

In a preferred embodiment R₁ or R₂ is 1H-pyrazolyl, more preferably 1H-pyrazol-2-yl or 1H-pyrazol-5-yl.

In a preferred embodiment one of R₁ and R₂ is thienyl, more preferably thien-2-yl.

R₃ is hydrogen, (C₁-C₃)alkyl, halogen or NO₂, more preferably R₃ can be methyl, ethyl, propyl, isopropyl, fluoro or NO₂, still more preferably it is methyl. In an advantageous form R₃ is a methyl in 4 position. When R₃ is halogen it is preferably fluoro in 4-position.

The compounds of the invention can be in form E and Z with reference to the definition of R₁ and R₂.

In a preferred embodiment the compound of formula (I) is selected from the group consisting of

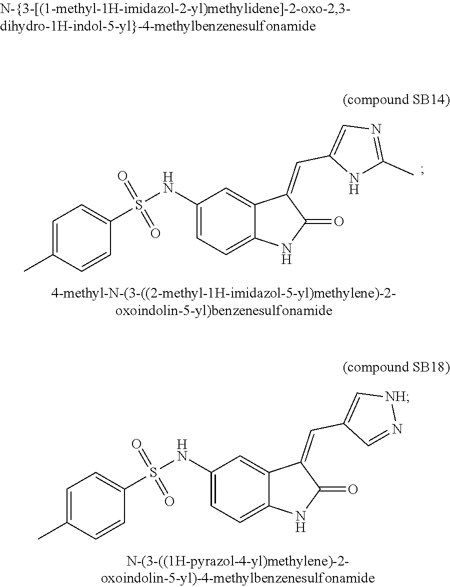

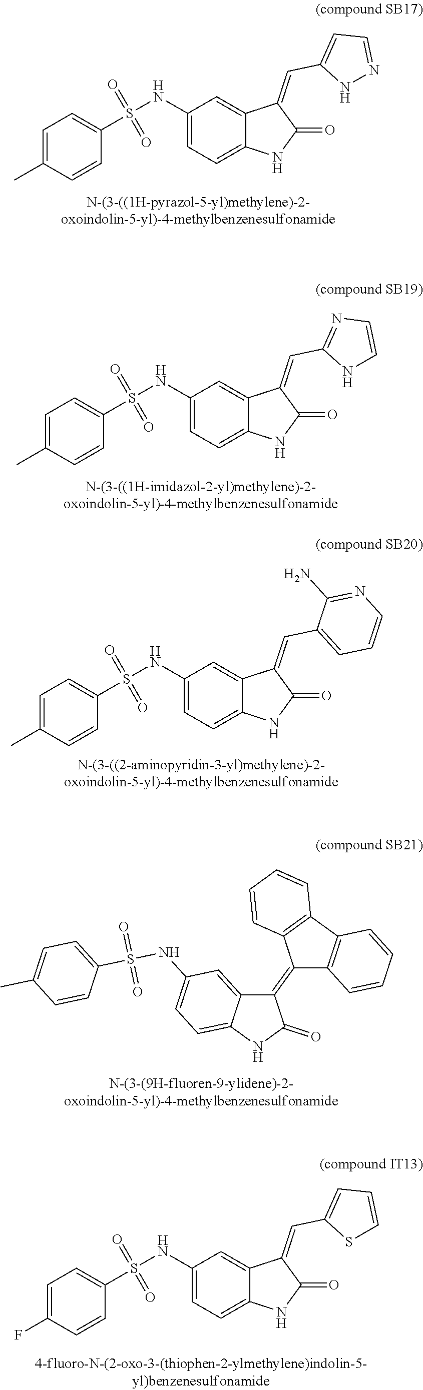 [SMILES: Cc1ccc(S(=O)(=O)Nc2ccc3c(c2)/C(=C/c2ccn[nH]2)C(=O)N3)cc1 ; Cc1ccc(S(=O)(=O)Nc2ccc3c(c2)/C(=C/c2ncc[nH]2)C(=O)N3)cc1 ; Cc1ccc(S(=O)(=O)Nc2ccc3c(c2)/C(=C/c2cccnc2N)C(=O)N3)cc1 ; Cc1ccc(S(=O)(=O)Nc2ccc3c(c2)C(=C2c4ccccc4-c4ccccc42)C(=O)N3)cc1 ; O=C1Nc2ccc(NS(=O)(=O)c3ccc(F)cc3)cc2/C1=C/c1cccs1]

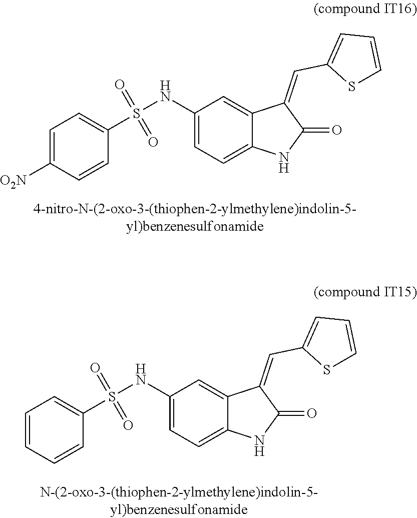 [SMILES: O=C1Nc2ccc(NS(=O)(=O)c3ccc([N+](=O)[O-])cc3)cc2/C1=C/c1cccs1 ; O=C1Nc2ccc(NS(=O)(=O)c3ccccc3)cc2/C1=C/c1cccs1]

In case of FC86 it can be in form E, i.e. N-[(3E)-2-oxo-3-(thiophene-2-ylmethylidene)-2,3-dihydro-1H-indol-5-yl]-4-methylbenzenesulfonamide) or in form Z, i.e. (N-[(3Z)-2-oxo-3-(thiophene-2-ylmethylidene)-2,3-dihydro-1H-indol-5-yl]-4-methylbenzenesulfonamide).

According to the present invention, hence all the compounds can be in E and Z forms, being comprised in the definitions all the stereo-compounds.

Therefore in a most preferred embodiment the invention relates a compound of Formula (I) selected from the group consisting of:
(Z)-4-methyl-N-(3-((2-methyl-1H-imidazol-5-yl)methylene)-2-oxoindolin-5-yl)benzenesulfonamide (compound SB14);
(E/Z)—N-(3-((1H-pyrazol-4-yl)methylene)-2-oxoindolin-5-yl)-4-methylbenzenesulfonamide (compound SB18);
(Z)—N-(3-((1H-pyrazol-5-yl)methylene)-2-oxoindolin-5-yl)-4-methylbenzenesulfonamide (compound SB17);
(Z)—N-(3-((1H-imidazol-2-yl)methylene)-2-oxoindolin-5-yl)-4-methylbenzenesulfonamide (compound SB19);
N-(3-((2-aminopyridin-3-yl)methylene)-2-oxoindolin-5-yl)-4-methylbenzenesulfonamide (compound SB20);
N-(3-(9H-fluoren-9-ylidene)-2-oxoindolin-5-yl)-4-methylbenzenesulfonamide (compound SB21);
(Z)-4-fluoro-N-(2-oxo-3-(thiophen-2-ylmethylene)indolin-5-yl)benzenesulfonamide (compound IT13);
(Z)-4-nitro-N-(2-oxo-3-(thiophen-2-ylmethylene)indolin-5-yl)benzenesulfonamide (compound IT16); and
(Z)—N-(2-oxo-3-(thiophen-2-ylmethylene)indolin-5-yl)benzenesulfonamide (compound IT15).

In a further aspect the invention concerns new 2-oxoindole compound or a pharmaceutically acceptable salt thereof for use as a medicament.

In an advantageous aspect, the new 2-oxoindole compound of Formula (I) or a pharmaceutically acceptable salt thereof can be used as a 14-3-3 protein modulator of a tumor. The tumor is preferably selected from the group consisting of a lymphoma, chronic lymphocytic leukemia (CLL), Ewing sarcoma, colon cancer, melanoma and anaplastic thyroid cancer (ATC).

Therefore, the invention concerns a new 2-oxoindole compound of Formula (I) or a pharmaceutically acceptable salt thereof for use as a 14-3-3 protein modulator of a tumor selected from the group consisting of a lymphoma, chronic lymphocytic leukemia (CLL), Ewing sarcoma, colon cancer, melanoma and anaplastic thyroid cancer (ATC).

Preferably the compounds of the invention are present in a pharmaceutical composition together with pharmaceutically acceptable carriers and excipients. The composition hence can comprise also pharmaceutically acceptable excipients and can be administered in a pharmaceutical form suitable for the desired administration route.

Pharmaceutically acceptable additives can be excipients, ligands, dispersing agents, colorants, humectants, commonly used for the preparation of tablets, capsules, pills, solutions, suspensions, emulsions for oral administration. Injectable solutions are also contemplated for parental administration, comprising subcutaneous, spinal and transdermal administration.

The compound of Formula (I) can be used as free base or in a salt form. Preferably, the salt is a salt selected from the group consisting of hydrochloride, hydrobromide, phosphate, sulphate, hydrogensulphate, alkylsulphonate, arylsulphonate, acetate, citrate, ossalate, maleate, fumarate, succinate, lactate, and tartrate. A salt can also be formed between a cation and a negatively charged group. Suitable cations include potassium ion, magnesium ion, calcium ion, and an ammonium cation such as tetramethylammonium ion. Examples of some suitable substituted ammonium ions are those derived from: ethylamine, diethylamine, triethylamine, ethanolamine, diethanolamine, piperazine, choline, meglumine, and tromethamine, as well as amino acids, such as lysine and arginine.

The pharmaceutical composition according to the present invention is preferably for intra-articular, intravenous, oral, transdermal, intrathecal, intranasal, intraperitoneal or intramuscular administration, more preferably oral administration.

The compound of Formula (I) of the invention is preferably in a dose in the range from 1 nM to 20000 nM, more preferably is in a dose in the range from 1 nM to 600 nM.

The invention will be now detailed with reference to the preparative examples of the compounds of the invention and examples for testing the antitumor activity with illustrative and not limitative purposes.

### EXPERIMENTAL PART

### Example 1 Preparation of the 2-Oxoindole Derivative Compounds

In the following experimental part, all the 2-oxoindole derivative compounds were prepared following the indications provided in “Synthesis of new enzyme inhibitors as potential tools for the antineoplastic therapy”-PhD thesis in Drug Science and Bioactive Substances-XXIV cycle-G. Nesi and described in Sestito et al. Eur J med Chem 2015 10.1016/j.ejmech.2015.10.020.

All the compounds (FC85, FC86, FC91, SB14, SB18, SB17, SB19, SB20, SB21, IT13, IT16 and IT15) were prepared following the synthetic procedure reported in the following scheme 2.

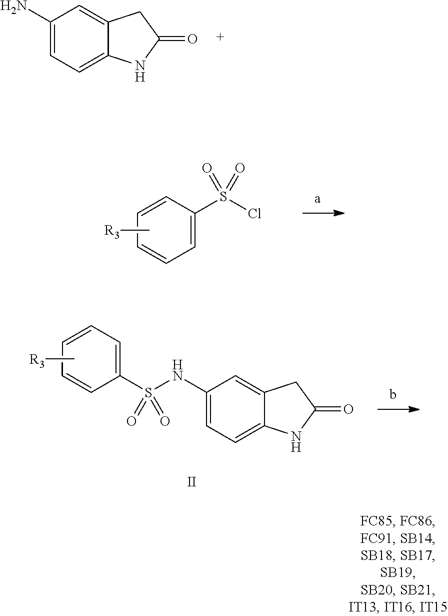

Reagents and Conditions: (a) H₂O, 20° C., 2 h; (b) appropriate carbaldehyde or ketone, EtOH, pirrolidine, 110° C., 16 h.

The condensation reaction of 5-amino-1,3-dihydro-2H-indol-2-one with the arylsulfonylchloride or with the mesyl chloride provided, respectively, N-(2-oxo-2,3-dihydro-1H-indol-5-yl)-arylsulfonamide and N-(2-oxo-2,3-dihydro-1H-indol-5-yl)methanesulfonamide. The products obtained were subjected to a condensation reaction with the appropriate aromatic carbaldehydes or fluorenone to provide the desired compounds.

The following 2-oxo-indole derivatives were then prepared:

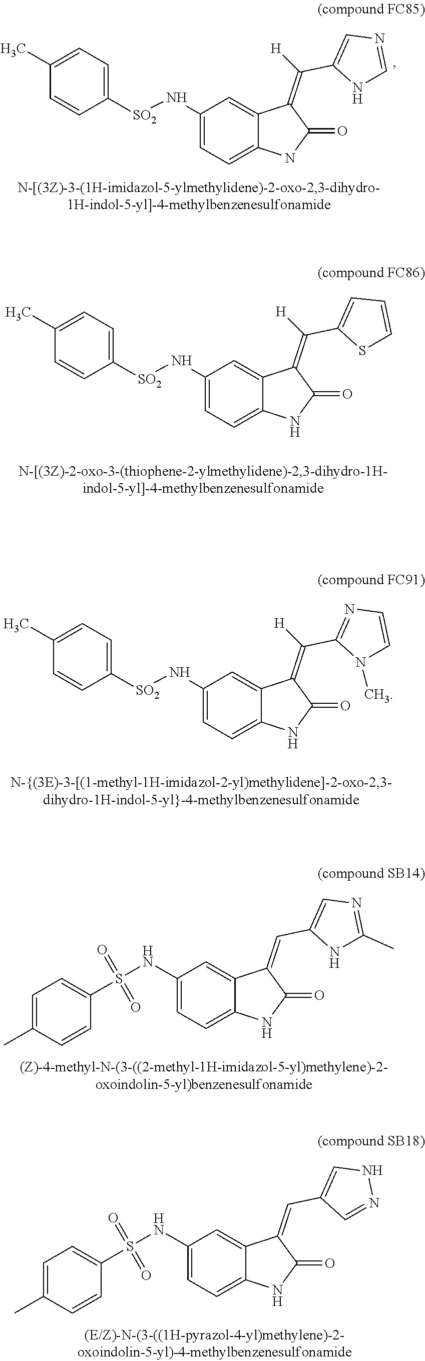

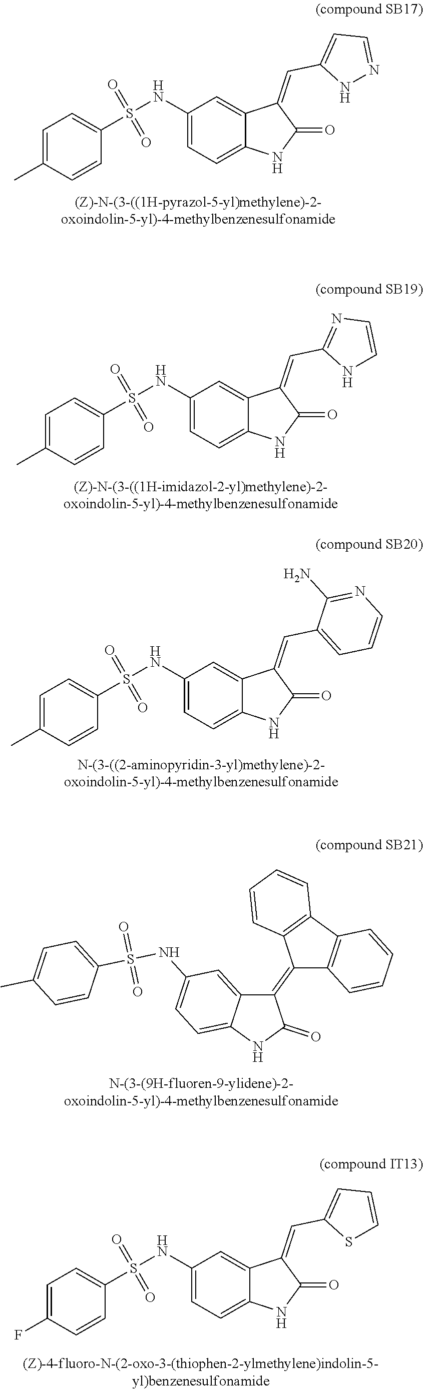 [SMILES: Cc1ccc(S(=O)(=O)Nc2ccc3c(c2)/C(=C/c2ccn[nH]2)C(=O)N3)cc1 ; Cc1ccc(S(=O)(=O)Nc2ccc3c(c2)/C(=C/c2ncc[nH]2)C(=O)N3)cc1 ; Cc1ccc(S(=O)(=O)Nc2ccc3c(c2)/C(=C/c2cccnc2N)C(=O)N3)cc1 ; Cc1ccc(S(=O)(=O)Nc2ccc3c(c2)C(=C2c4ccccc4-c4ccccc42)C(=O)N3)cc1 ; O=C1Nc2ccc(NS(=O)(=O)c3ccc(F)cc3)cc2/C1=C/c1cccs1]

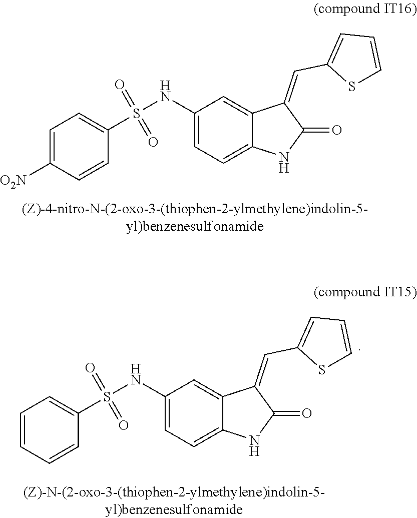 [SMILES: O=C1Nc2ccc(NS(=O)(=O)c3ccc([N+](=O)[O-])cc3)cc2/C1=C/c1cccs1 ; O=C1Nc2ccc(NS(=O)(=O)c3ccccc3)cc2/C1=C/c1cccs1]

### Example 2 Experimental Protocols

### Chemistry.

The solvents were all acquired from Sigma-Aldrich/Merck to an analytical degree of purity (>99%). Commercial chemical reagents were acquired from Sigma-Aldrich/Merck, Fluorochem, Tokyo Chemical Industry (TCI), or Alfa Aesar and used without further purification. The structure of the compounds and their degree of purity (>95%) were checked by means of 1H-NMR and 13C-NMR spectrometry.

The anhydrous environment, necessary in some sensitive reactions, was obtained using nitrogen atmosphere. The evaporations were carried out in a rotary vacuum evaporator PC3001 VARIOpro, and sodium sulphate (Na2SO4) was used as the dehydration agent. Reaction monitoring was performed by Thin Layer Chromatography (TLC) using 60F254 (Sigma-Aldrich/Merck) silica gel plates adsorbed on aluminum supports. TLCs were visualized using a UV lamp (Short wave: λmax=254 nm; Long wave λmax=365 nm), and or PMA (10% phosphomolybdic acid in EtOH), and or DNP (6% 2,4-dinitrophenylhydrazine, 60% 1 M H2SO4 in EtOH).

Microwave reactions (MW) were performed using a Biotage® Initiator+microwave. Filtrations were performed using Celite® 545 (Sigma Aldrich) as the filtering agent. Flash chromatographic column purifications were performed using high purity silica gel: 40-63 μm (Sigma-Aldrich/Merck). Alternatively, it was performed automatically using the Biotage® Isolera™ Prime instrument. The 1H-NMR and 13C-NMR spectra were recorded by Bruker Avance 400 instrument, respectively at 400 and 101 MHz. The chemical shifts (6) were expressed in ppm using the residual solvent as internal standard (1H-NMR: CDCl3, 7.26; D2O, 4.79; CD3OD, 3.31; DMSO-d6, 2.50; 13C-NMR: CDCl3, 77.0; CD3OD, 49.0; DMSO-d6, 39.5). The coupling constants (J) are reported in Hz with the following abbreviations: s, singlet; d, doublet; t, triplet; q, quartet; m, multiplet; br s: broad signal.

### General Procedure for the Synthesis of SB14, SB17, SB18, SB19, SB20, SB21, IT15, IT16, IT13

In a vial, the appropriate arylsulfonamide II (0.165 mmol) is dissolved in EtOH, and then the appropriate carbaldehyde or ketone (0.182 mmol) and a catalytic amount of piperidine are added. The reaction was then stirred at 110° C. for 16 h or at 140° C. for 10 min in the microwave reactor. After this period, the solvent is removed and the crude obtained was triturated with MeOH. Subsequently, the solid obtained was filtered, giving the desired compound.

### SB14: (Z)-4-methyl-N-(3-((2-methyl-1H-imidazol-5-yl)methylene)-2-oxoindolin-5-yl)benzenesulfonamide

Yield: 18%; 1H NMR (DMSO-d6): δ 2.32 (s, 3H, CH3); 2.45 (s, 3H, CH3); 6.70-6.80 (m, 2H, Ar); 7.30-7.38 (m, 3H, Ar); 7.57-7.66 (m, 4H, Ar); 9.90 (br s, 1H, NH); 10.91 (br s, 1H, NH) ppm.

### SB18: (E/Z)—N-(3-((1H-pyrazol-4-yl)methylene)-2-oxoindolin-5-yl)-4-methylbenzenesulfonamide (Mix 40:60)

Yields: 16%

1HNMR (MeOD): δ 2.33-2.35 (m, 3H, CH3); 6.70 (d, 1H, J=8 Hz, Ar, Z-isomer); 6.76-6.82 (m, 2H, Ar); 6.90 (dd, 1H, J=8, 1.6 Hz, Ar, Z-isomer); 7.28 (d, 2H, J=8 Hz, Ar); 7.32 (d, 1H, J=1.6 Hz, Ar); 7.65-7.61 (m, 3H, Ar e CH═); 8.02 (br s, 1H, Ar); 8.61 (br s, 1H, NH) ppm.

### SB17: (Z)—N-(3-((1H-pyrazol-5-yl)methylene)-2-oxoindolin-5-yl)-4-methylbenzenesulfonamide

Purified by chromatography eluting with AcOEt/EP 6:4. Yields: 5%; 1H NMR (MeOD): δ 2.30 (s, 3H, CH3); 6.69 (d, 1H, J=2.4 Hz, Ar); 6.74 (d, 1H, J=8 Hz, Ar); 6.88-6.90 (m, 1H, Ar); 7.24 (d, 2H, J=8.2 Hz, Ar); 7.57 (s, 1H, CH═); 7.60 (d, 2H, J=8.2 Hz, Ar), 7.79 (br s, 1H, Ar); 8.67 (br s, 1H, Ar) ppm.

13C NMR (MeOD): δ 147.9; 145.0; 144.9; 141.2; 139.5; 139.4; 137.9; 137.7; 132.9; 130.6; 130.5; 129.0; 126.5; 123.6; 116.6; 112.7; 111.4; 110.7; 21.4 ppm.

### SB19: (Z)—N-(3-((1H-imidazol-2-yl)methylene)-2-oxoindolin-5-yl)-4-methylbenzenesulfonamide

Yields: 70%; 1H NMR (DMSO): δ 2.32 (s, 3H, CH3); 6.76 (d, 1H, J=8.2 Hz, Ar); 6.88 (dd, 1H, J=1.8, 8.2 Hz, Ar); 7.33 (d, 2H, J=8.2 Hz, Ar); 7.35 (s, 1H, CH═); 7.43 (d, 1H, J=1.6 Hz, Ar); 7.55-7.56 (m, 2H, Ar); 7.61 (d, 2H, J=8.2 Hz, Ar) ppm.

### SB20: N-(3-((2-aminopyridin-3-yl)methylene)-2-oxoindolin-5-yl)-4-methylbenzenesulfonamide

Purified by column chromatography with gradient elution mode using CH3Cl/ACN/0.1% Et3N (9:1:0.1 to 6:4:0.1)

Yields: 57%;

SB20A (Z-isomer): 1HNMR (MeOD): δ 2.37 (s, 3H, CH3); 6.66-6.71 (m, 1H, Py); 6.77 (d, 1H, J=8.2 Hz, Ar); 6.95 (dd, 1H, J=8.2, 1.6 Hz); 7.11 (d, 1H, J=1.6 Hz, Ar); 7.25 (d, 2H, J=8 Hz, Ar); 7.47 (d, 2H, J=8 Hz, Ar); 7.50 (s, 1H, CH═), 7.59-7.61 (m, 1H, Py); 8.06-8.07 (m, 1H, Py) ppm.;

SB20B (E-isomer): 1HNMR (MeOD): δ 2.37 (s, 3H, CH3); 6.66-6.71 (m, 2H, Py e Ar); 6.81 (dd, 1H, J=8.2, 1.6 Hz); 7.29 (d, 2H, J=8 Hz, Ar); 7.43 (d, 1H, J=1.6 Hz, Ar); 7.47 (d, 2H, J=8 Hz, Ar); 7.53 (s, 1H, CH═), 7.96-7.98 (m, 1H, Py); 8.38-8.40 (m, 1H, Py) ppm.

### SB21: N-(3-(9H-fluoren-9-ylidene)-2-oxoindolin-5-yl)-4-methylbenzenesulfonamide

Purified by column chromatography with gradient elution mode using EP/AcOEt (from 8:2 to 6:4),

Yield: 52%; 1H NMR (MeOD): δ 2.37 (s, 3H, CH3); 6.77 (d, 1H, J=8.2 Hz, Ar); 6.90-6.92 (m, 1H, Ar); 7.14 (t, 1H, J=7.6 Hz, Ar); 7.20 (t, 1H, J=7.6 Hz, Ar), 7.31 (d, 2H, J=8 Hz, Ar); 7.36-7.42 (m, 2H, Ar); 7.59 (d, 2H, J=8 Hz, Ar); 7.64-7.69 (m, 2H, Ar); 7.81 (br s, 1H, Ar); 8.13 (d, 1H, J=8 Hz, Ar); 8.95 (d, 1H, J=8 Hz, Ar) ppm.

13C NMR (MeOD): δ 150.8; 145.0; 144.3; 143.1; 141.8; 138.8; 138.1; 137.9; 132.9; 132.7; 132.2; 130.6; 128.9; 128.8; 128.7; 128.3; 127.6; 125.0; 121.2; 120.6; 111.4; 21.44 ppm.

### IT15 (E/Z) N-[(3)-2-oxo-3-[(thiophen-2-yl)methylidene]-2,3-dihydro-1H-indol-5-yl]benzenesulfonamide

Yields 58%; 1H NMR (Acetone) δ 6.77-6.78 (d, 1H, J=8.4 Hz, Ar) 6.89-6.94 (dd, 1H, J=8.4, 2 Hz, Ar) 7.21-7.23 (m, Ar, isom-E) 7.28-7.31 (m, Ar, isom-Z) 7.52-7.53 (m, 2H, Ar) 7.83-7.84 (d, 1H, J=5.2 Hz, Ar) 7.80-7.98 (m, 4H) 8.36-8.38 (d, 2H, J=8.8 Hz, Ar) ppm

### IT16: (E/Z) 4-nitro-N-[(3E)-2-oxo-3-[(thiophen-2-yl)methylidene]-2,3-dihydro-1H-indol-5-yl]benzene-1-Sulfonamide

Yields 64%; 1H NMR (Acetone) δ 6.80-6.82 (d, 1H, J=8.4 Hz) 6.90-6.93 (dd, 1H, J=8.4, 2 Hz) 7.21-7.29 (m, 1H, Ar) 7.56-7.57 (d, 1H, J=2 Hz, Ar) 7.82-7.84 (d, 1H, J=5.2 Hz, Ar) 7.99-7.80 (m, 4H) 8.36-8.38 (d, 2H, J=8.8 Hz, Ar) ppm

### Example 3: Evaluation of the Compounds of the Invention

The following compounds were tested in lymphoma cell lines:

| sigla | Struttura |
|---|---|
| FC85 | 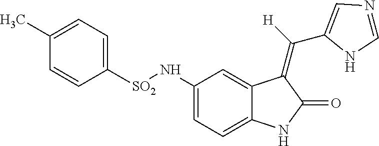 [SMILES: Cc1ccc(S(=O)(=O)Nc2ccc3c(c2)/C(=C/c2cnc[nH]2)C(=O)N3)cc1] |
| FC86 | 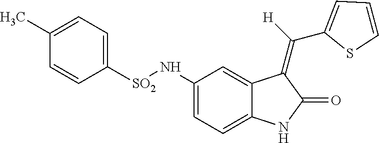 [SMILES: Cc1ccc(S(=O)(=O)Nc2ccc3c(c2)/C(=C/c2cccs2)C(=O)N3)cc1] |
| FC91 | 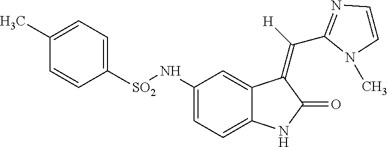 [SMILES: Cc1ccc(S(=O)(=O)Nc2ccc3c(c2)/C(=C/c2nccn2C)C(=O)N3)cc1] |
| FC96 | 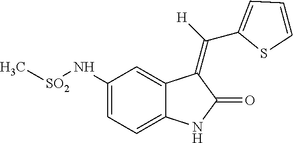 [SMILES: CS(=O)(=O)Nc1ccc2c(c1)/C(=C/c1cccs1)C(=O)N2] |
| FC100 | 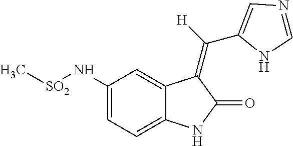 [SMILES: CS(=O)(=O)Nc1ccc2c(c1)/C(=C/c1cnc[nH]1)C(=O)N2] |

FC96 and FC100 as comparison compounds were prepared and described in Sestito et al. Eur J med Chem 2015 10.1016/j.ejmech.2015.10.020 as also having activity against pancreas tumor.

All the above compounds were tested in vitro for their anticancer activity against a panel of eight B-cell lymphoma cell lines by MTT proliferation assay. The tested cell lines belonging to mantle cell lymphoma (MCL: REC1, Z138), activated B-cell diffuse large B-cell lymphoma (ABC-DLBCL: R11, HBL1, U2932, TMD8), germinal center B-cell diffuse large B-cell lymphoma (GCB-DLBCL: SU-DHL-10, FARAGE) subtypes. In overall, MCL cell lines present Cyclin D1 overexpression, while ABC- and GCB-DLBCL's proliferation is BCR and BCL6/HDACs driven, respectively. At the RNA level, all tested lymphoma cell lines were characterized by an overexpression of 14-3-3 zeta as reported for B-cell lymphomas resistant to chemotherapeutic regimen. The IC₅₀ calculated after 72 h of drug incubation are reported in Table 1.

TABLE 1
IC₅₀ values expressed in nM

| | Cell Lines | | | | | | | |
|---|---|---|---|---|---|---|---|---|
| Compound | RI1 | HBL1 | U2932 | TMD8 | SUDHL10 | FARAGE | Z138 | REC1 |
| FC96 | 20000 | R | R | R | R | R | R | 15000 |
| FC100 | 5500 | R | R | R | 19000 | R | 8000 | 16000 |
| FC85 | 500 | 600 | 500 | 600 | 600 | 500 | 500 | 1000 |
| FC86 | 25 | 25 | 20 | 20 | 25 | 15 | 25 | 10 |
| FC91 | 30 | 40 | 50 | 50 | 50 | 25 | 50 | 50 |

R, resistant cell line (IC50 >> 20000 nM), n/a, not available

Data collected proved that FC85, FC86 and FC91 were the most active compounds with IC₅₀<1 μM in all eight lymphoma cell lines, whereas FC96 and FC100 proved to be almost inactive.

All data collected proved that compounds FC86 and FC91 showed IC₅₀ in the nanomolar range in eight different lymphoma cell lines.

### Example 4: Evaluation of the Activity of FC86 and Analogs in ATC Cell Lines

The compounds FC91, SB14, SB17, SB18, SB19, SB20, SB21 and FC85 were employed in a cellular screening at fixed concentration ranging from 20 nM to 20 μM. FIG. 1 shows the antiproliferative activity of FC86 in four human ATC cell lines. Table 2 reports the percentage of proliferating activity of four human ATC cell lines after the treatment with molecules object of this patent at fixed concentrations of 20 nM, 100 nM and 2000 nM Notably, compounds FC91, SB18 and SB19 showed IC₅₀ values comprised between 100 and 200 nM, whereas compounds SB14, SB17, SB18, SB19, SB20, SB21 and FC85 showed IC₅₀ value comprised between 100-20000 nM in one (i.e. SB17) to four (i.e. SB20b) cell lines of anaplastic thyroid cancer which is a rare, highly aggressive malignant tumor accounting for 2 to 3 percent of all thyroid gland neoplasms.

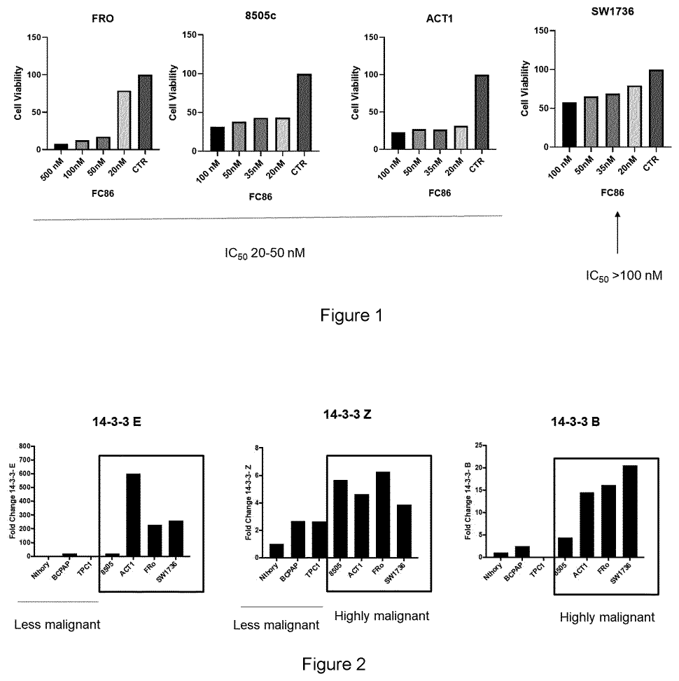

Noteworthy, the four human ATC cell lines Nthory, 8505c, SW1736 and FRO were analysed for the expression of 14-3-3 isoforms. As reported in FIG. 2 at the RNA level, malignant anaplastic thyroid cancer (ATC) cell lines are characterized by an overexpression of 14-3-3 Z, E and B in comparison with less malignant papillary thyroid cancer cell lines. This result confirm that compounds of the invention were modulators of 14-3-3 protein

### Example 5. Toxicity Evaluation in Normal Rat and Human Thyroid Cancer Cell Lines

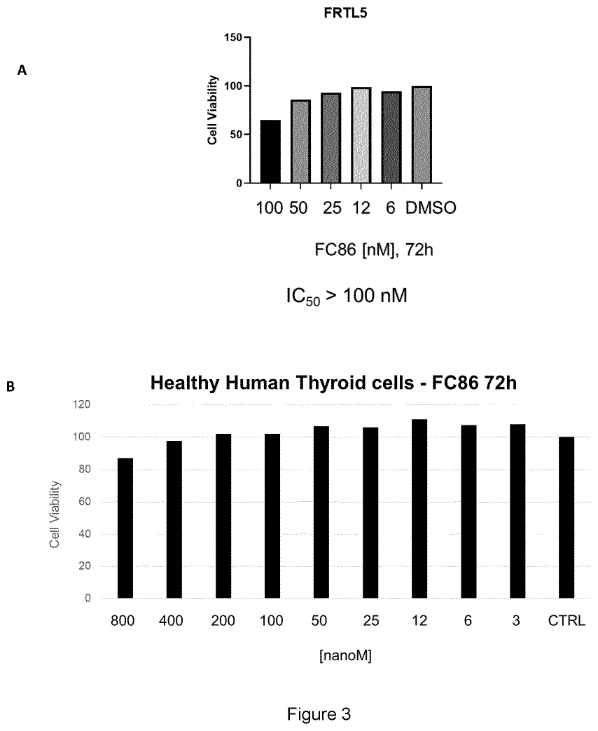

To assess the toxicity FC86 was evaluated against a primary healthy human thyroid cell line at concentrations ranging from 3 nM to 800 nM for 72 h (FIG. 3). Data collected reveal that FC86 is not toxic for healthy human thyroid cells at concentration higher than 400 nM. Such a concentration is 20× fold higher than IC₅₀ detected in tumour cell lines.

The following table contains data of cell viability determined in % at the indicated concentrations on four cell lines of human ATC.

Table 2: Four human thyroid cancer cell lines were incubated with FC91, SB14, SB17, SB18, SB19, SB20A, SB20b, SB21 and FC85 for 48 h at fixed concentration of 20,100 and 20000 nM and then assayed with MTT. Data are reported as % of proliferating cells.

| | 20 nM | | | | 100 nM | | | | 20000 nM | | | |
|---|---|---|---|---|---|---|---|---|---|---|---|---|
| | Nthory | 8505c | SW1736 | FRO | Nthory | 8505c | SW1736 | FRO | Nthory | 8505c | SW1736 | FRO |
| FC91 | 84 | 79 | 73 | 63 | 55 | 38 | 52 | 46 | 47 | 48 | 60 | 55 |
| SB14 | 95 | 97 | 84 | 92 | 77 | 128 | 102 | 113 | 41 | 65 | 70 | 57 |
| SB17 | 89 | 90 | 81 | 80 | 73 | 119 | 99 | 113 | 48 | 59 | 63 | 55 |
| SB18 | 54 | 80 | 77 | 77 | 58 | 48 | 64 | 54 | 34 | 42 | 59 | 56 |
| SB19 | 81 | 74 | 80 | 78 | 61 | 74 | 85 | 74 | 43 | 37 | 59 | 51 |
| SB20A | 99 | 78 | 82 | 85 | 77 | 99 | 89 | 107 | 63 | 35 | 60 | 55 |
| SB20B | 95 | 75 | 88 | 78 | 74 | 101 | 102 | 85 | 46 | 24 | 25 | 29 |
| SB21 | 91 | 82 | 108 | 93 | 94 | 134 | 103 | 114 | 15 | 37 | 64 | 60 |
| FC85 | 78 | 80 | 90 | 75 | 83 | 99 | 67 | 98 | 38 | 36 | 56 | 42 |

The percentage of proliferating cells after the treatment of four Human ATC cell lines has been hence calculated with compounds FC91, SB14, SB17, SB18, SB19, SB20A, SB20b, SB21 and FC85 for 48 h. The viability of cells >80% showed that tested compounds were not able to exert the antiproliferative activity, whereas the viability of cells <60% indicates a significant antiproliferative activity of tested compounds at fixed concentration of 20, 100 and 20000 nM. Data indicate that at 20000 nM all the compounds exerted a significant antiproliferative activity.

### Example 6: Evaluation of Activity of FC86

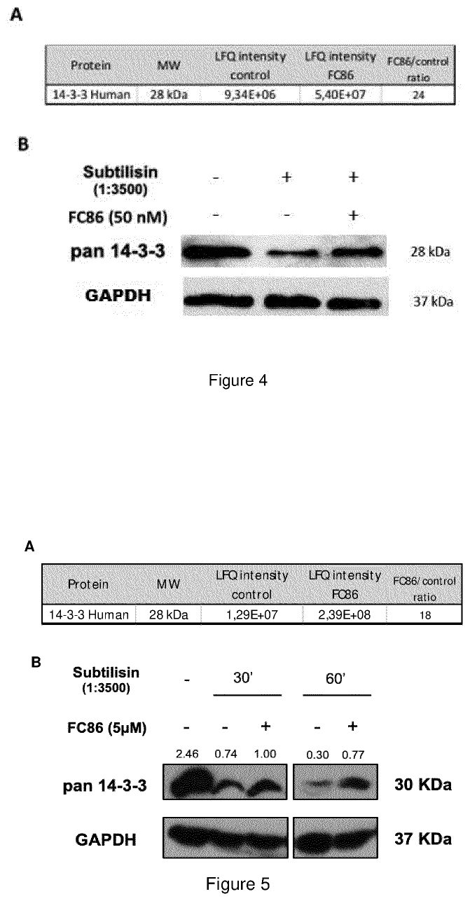

Despite the chemical structure of FC86 deposes for an activity against some kinases, the profiling done by ProQinase (at 1 and 0.1 μM) on 410 human kinases revealed the lack of activity against any of them. Based on these results and to uncover its mechanism of action, the inventors have pursued a campaign of target identification, performing a proteomic-based study [Lomenick B, Jung G, Wohlschlegel J A, Huang J: Target identification using drug affinity responsive target stability (DARTS). Current protocols in chemical biology 2011, 3:163-180]. In particular, DARTS experiments were performed by incubating lymphoma cells (U937) with sub-toxic amounts of FC86 (50 nM), leading to the identification of 14-3-3 as the only protein significantly stabilized by the compound. Indeed, when protein lysates from treated and untreated cells were subjected to a partial subtilisin-catalyzed digestion, a significant protection from proteolysis was observed for 14-3-3 from the cells incubated with FC86. This result was obtained in several experiments analyzed by both mass spectrometry and western blot (FIG. 4). The same DARTS experiments were also performed by incubating glioblastoma cell lines (U87MG) with subtoxic amounts of FC86 and data collected confirmed the 14-3-3 as target protein. (FIG. 5).

### Example 7 Early Toxicity Profiling

Since the progression in drug discovery of small molecules is strongly limited by the ability to modulate known off-targets (or anti-targets), the inventors evaluated the capability of the compounds to induce undesirable side-effects.

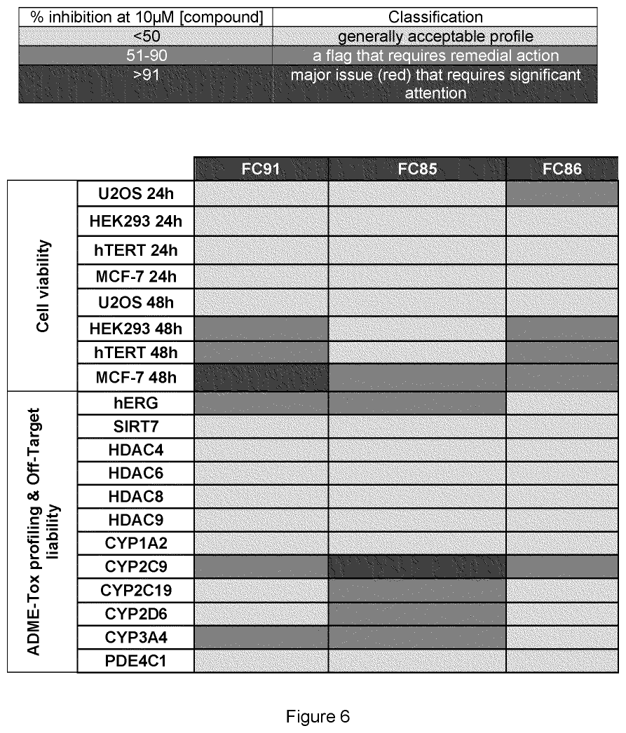

To assess the ADME-TOX profile of selected compounds (FC91, FC86, FC85, a full panel of in vitro assays was performed as reported in Runfola et al. [Runfola M, Sestito S, Gul S, Chiellini G, Rapposelli S: Collecting data through high throughput in vitro early toxicity and off-target liability assays to rapidly identify limitations of novel thyromimetics. Data in brief 2020, 29:105206.]. Cytotoxic effects were evaluated on 4 different cell lines (U2OS, hTERT, HEK293, MCF-7) at 24 h and 48 h after treatment with tested compounds. Cardiac toxicity was assessed evaluating the inhibition effect of compounds on the hERG ion-channel, which is responsible of the withdrawn of several drugs in clinical trials. Metabolic interference was evaluated testing inhibitory effects of new compounds on five isoforms of CYP450 (CYP1A2, CYP2C9, CYP2C19, CYP2D6, CYP3A4). To assess off-target liabilities, the inhibitory effects have been assessed against several well-known off-targets. In particular, HDAC4, HDAC6, HDAC8, HDAC9, and SIRT7 were chosen as representatives of an undesirable epigenetic modulation. Moreover, compounds were screened against phosphodiesterase (PDE4C1), known for its role in cell survival and tumorigenesis. All screening experiments were performed at a concentration of 10 μM (triplicate) where the results for each compound were normalized using the respective raw data to high and low controls. The data were collected and analyzed in the form of a traffic-light system using criteria shown below. Results are reported in FIG. 6.

FC86 proved to be safe, and it did not show inhibitory effect on CYP450 isoforms. On this basis, the inventors decided to move on with the evaluation of the anticancer activity in tumors cells characterized by the overexpression of 14-3-3 protein [Challen G A, Martinez G, Davis M J, Taylor D F, Crowe M, Teasdale R D, Grimmond S M, Little M H: Identifying the molecular phenotype of renal progenitor cells. Journal of the American Society of Nephrology: JASN 2004, 15:2344-2357.].

The anticancer activity was hence investigated in colon cancer, five Ewing sarcoma cell lines. The antiproliferative activity was assessed by MTT proliferation assay (Table III) The anticancer activity was also investigated in a rare and lethal cancer (i.e. anaplastic thyroid cancer) by the use of three ATC-derived cell lines (Sw, FRO, 8505c). Again, the IC50 value after 72 h exposure was in the range of 10 to 50 nM. The results are reported in Table 3. The same trend was also observed in four melanoma cell lines A2058, M14 (genotype BRAFV600E), SKMEL 197 e MEWO (genotype BRAF wild-type). IC₅₀ values are reported in Table 4.

Table III IC₅₀ expressed in μM and Emax (at 30 μM) for FC86 after 72 h exposure, were calculated by MTT assay

TABLE 3

FC86

| Cell line | | IC50 (μM) | Emax (%) at 30 μM |
|---|---|---|---|
| Colon | HCT-116 | 1.6 | 62 |
| SCLC | NCI-H69 | 1.4 | 80 |
| SCLC | NCI-H82 | 3.0 | 55 |
| Ewing Sarcoma | TC-32 | 0.03 | 75 |
| Ewing Sarcoma | SKNMC | >30 | n.d |
| Ewing Sarcoma | 6647 | 0.09 | 75 |
| Ewing Sarcoma | TC-71 | 0.8 | 95 |
| Ewing Sarcoma | A673 | 3.2 | 85 |

TABLE 4
IC₅₀ values for four different melanoma cell lines. The data show
the value of IC50 and the respective 95% confidence interval.

| Melanoma cell lines | IC50 (nM) |
|---|---|
| A2058 | 28.57 (23.65-35.24) |
| M14 | 14.08 (10.34-18.77) |
| MEWO | 57.04 (46.46-70.09) |
| SKMEL 197 | 20.73 (16.63-25.49) |

Data collected proved that FC86 elicits a significant antiproliferative activity against colon cancer Ewing Sarcoma and SCLC. In particular, it showed a potency in the nanomolar range in 8 lymphoma cell lines, in 4 Ewing sarcoma, in 4 different ATC cancer cell lines and in 4 different melanoma cell lines.

### Example 8 In Vivo Antiproliferative Activity

The promising in vitro data suggests a deeper investigation of FC86. To this purpose, the compound was administered by oral gavage to Nod-Scid mice engrafted with the human lymphoma cell line TMD8 (15 millions of cells in 100 uL PBS/mouse). FC86 given once daily and five times per week at the dose of 100 mg/kg was able to significantly inhibit tumor growth (p<0.01). At the end of the study, the tumor receiving FC86 were two times smaller than those receiving only the vehicle (FIG. 7). Body conditions were not affected by the treatments.

### Example 9 Drug Toxicity Evaluation on Primary Healthy Cells

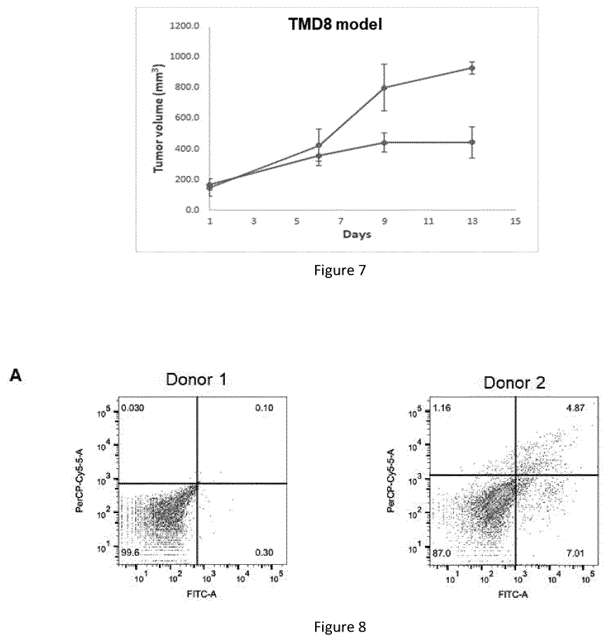

Since the drug treatment did not have any toxic effect on the body condition of the mice, the inventors were also interested to verify the lack of toxicity in primary cells. Hence, the inventors attempted to verify the effects of FC86 on peripheral blood mononuclear cells (PBMC), isolated from healthy donors. PBMC were treated with 200 nM of FC86 (10× the IC₅₀ values reached in lymphoma cell lines) and analyzed for the activation of Annexin V (marker of apoptosis) (FIG. 8) as final read-out, the inventors did not detect any sign of apoptosis after 48 h of drug incubation

### Example 10

Drug Affinity Responsive Target Stability (DARTS) test

The identification of putative targets of FC86 was achieved by Drug Affinity Responsive Target Stability (DARTS) experiments. Cancer cells were incubated with a sub-toxic concentration (50 nM for U937 and 5 μM for U87MG) of FC86 for 2 h or with the medium (control cells). After the treatments, treated and control cells were collected and whole protein extracts were prepared by lysing cells in 20 mM Tris-HCl (pH 7.5), 150 mM NaCl, 1 mM EDTA, 1 mM EGTA, 1% NP-40, 1% sodium deoxycholate, 2.5 mM sodium pyrophosphate, 1 mM β-glycerophosphate, 1 mM Na3VO4, 1 μg/ml leupeptin. Protein concentration was determined by the Bradford protein assay using bovine serum albumin as standard. Identical amounts of proteins (50 μg) from each sample were subjected to a limited digestion with subtilisin (protein/enzyme ratio 1:2500 w/w, 30 min, 25° C.). The resulting partially hydrolyzed protein mixtures were separated by 10% SDS-PAGE. To get protein identification, the gels were divided in 10 pieces, and each underwent to a trypsin in gel digestion procedure. NanoUPLC-hrMS/MS analyses of the resulting peptides mixtures were carried out on a Q-Exactive orbitrap mass spectrometer (Thermo Fisher), coupled with a nanoUltimate300 UHPLC system (Thermo Fisher). Peptides were separated on a capillary BEH C18 column (0.075 mm×100 mm, 1.7 μm, Waters) using aqueous 0.1% formic acid (A) and CH3CN containing 0.1% formic acid (B) as mobile phases and a linear gradient from 5% to 50% of B in 90 minutes and a 300 nL min-1 flow rate. Mass spectra were acquired over an m/z range from 400 to 1800. To achieve protein identification, MS and MS/MS data underwent Mascot software (Matrix Science) analysis using the non-redundant Data Bank UniprotKB/Swiss-Prot (Release 2020_03). Parameters sets were: trypsin cleavage; carbamidomethylation of cysteine as a fixed modification and methionine oxidation as a variable modification; a maximum of two missed cleavages; false discovery rate (FDR), calculated by searching the decoy database, 0.05. A comparison between the proteins found in the different samples allowed discriminating those proteins remained partially undigested in the compound-treated cells and largely hydrolyzed in the untreated control cells; those proteins were considered putative targets. The same procedure was also performed using western blot as a detection method and using anti-14-3-3 and anti GAPDH antibodies.

1. A method for the treatment of a tumor comprising the step of administering a 2-oxoindole compound of Formula (I) or a pharmaceutically acceptable salt thereof 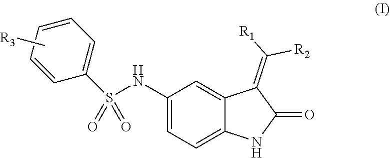
wherein
R₁ and R₂ are, independently from each other, hydrogen, 1H-imidazolyl, thienyl and 1-methyl-1H-imidazolyl, 2-methyl-1H-imidazolyl, 2-aminopyridinyl, 1H-pyrazolyl, with the proviso that one of R₁ and R₂ is hydrogen; or R₁ and R₂ together form 9H-fluorene
R₃ is hydrogen, (C₁-C₃)alkyl, halogen or NO₂;
wherein the tumor is selected from the group consisting of a lymphoma, chronic lymphocytic leukaemia (CLL), Ewing sarcoma, colon cancer melanoma and anaplastic thyroid cancer (ATC).

2. The method of claim 1, wherein one of R₁ and R₂ is thienyl.

3. The method of claim 1 wherein one of R₁ and R₂ is 1H-pyrazolyl, preferably 1H-pyrazol-2-yl or 1H-pyrazol-5-yl.

4. The method of claim 1 wherein one of R₁ and R₂ is 1H-imidazolyl, preferably 1H-imidazol-2-yl or 1H-imidazolyl-5-yl.

5. The method of claim 1, wherein R₃ is methyl.

6. The method of claim 1 wherein the compound of formula (I) or a pharmaceutically acceptable salt thereof is selected from the group consisting of:

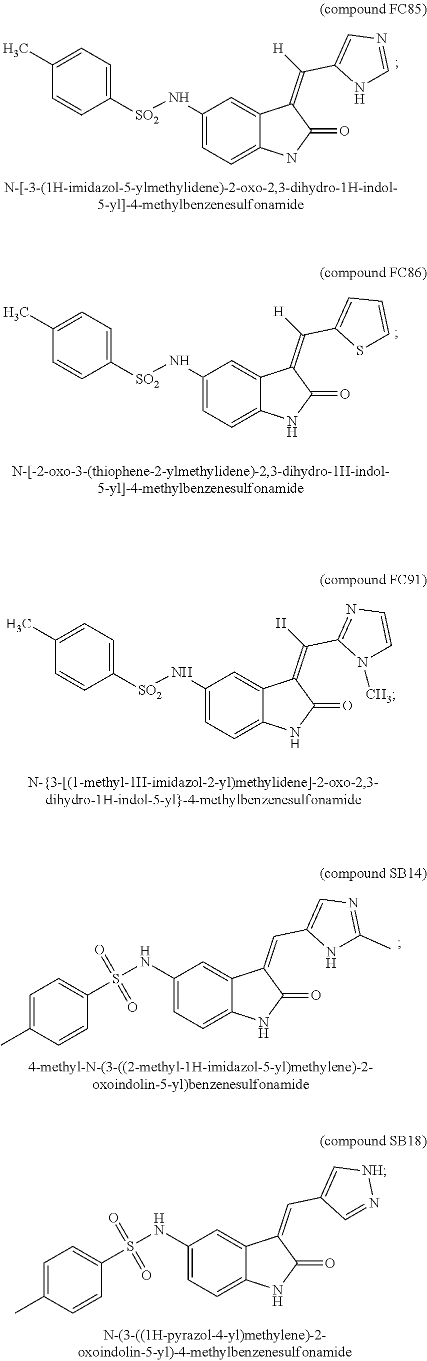

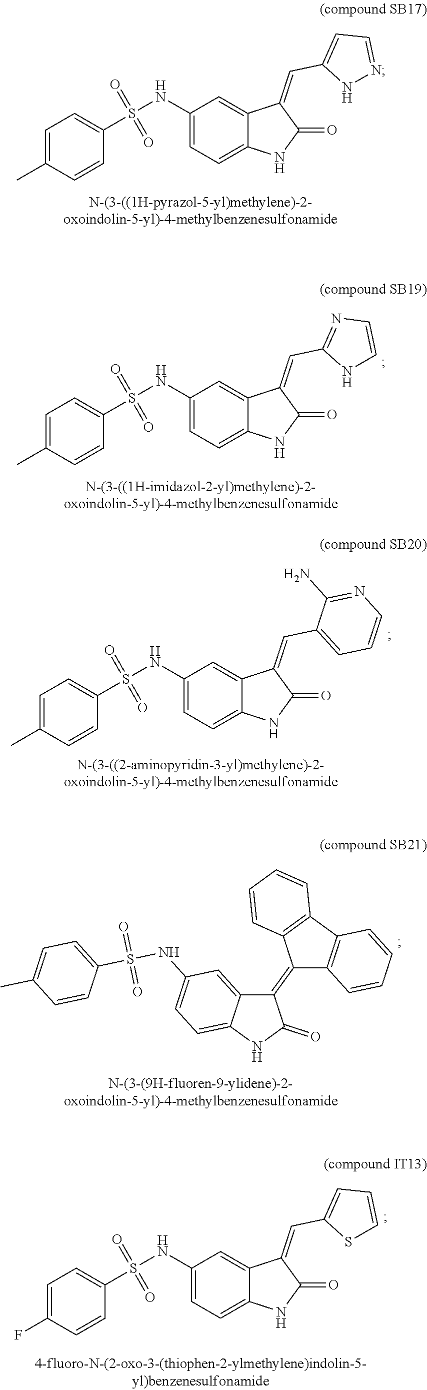 [SMILES: Cc1ccc(S(=O)(=O)Nc2ccc3c(c2)/C(=C/c2ccn[nH]2)C(=O)N3)cc1 ; Cc1ccc(S(=O)(=O)Nc2ccc3c(c2)/C(=C/c2ncc[nH]2)C(=O)N3)cc1 ; Cc1ccc(S(=O)(=O)Nc2ccc3c(c2)/C(=C/c2cccnc2N)C(=O)N3)cc1 ; Cc1ccc(S(=O)(=O)Nc2ccc3c(c2)C(=C2c4ccccc4-c4ccccc42)C(=O)N3)cc1 ; O=C1Nc2ccc(NS(=O)(=O)c3ccc(F)cc3)cc2/C1=C/c1cccs1]

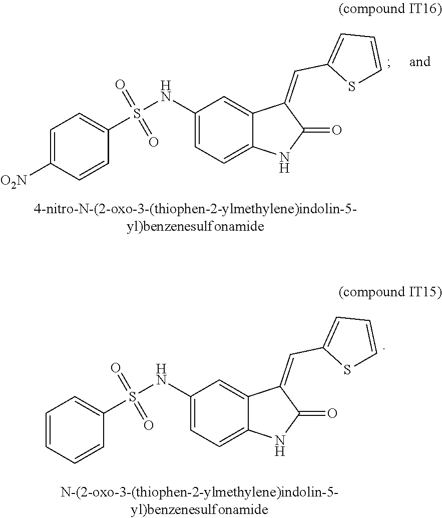 [SMILES: O=C1Nc2ccc(NS(=O)(=O)c3ccc([N+](=O)[O-])cc3)cc2/C1=C/c1cccs1 ; O=C1Nc2ccc(NS(=O)(=O)c3ccccc3)cc2/C1=C/c1cccs1]

7. The method of claim 1 wherein the compound of formula (I) or a pharmaceutically acceptable salt thereof is selected from the group consisting of:
N-[(3Z)-3-(1H-imidazol-5-ylmethylidene)-2-oxo-2,3-dihydro-1H-indol-5-yl]-4-methylbenzenesulfonamide (compound FC85);
N-[(3Z)-2-oxo-3-(thiophene-2-ylmethylidene)-2,3-dihydro-1H-indol-5-yl]-4-methylbenzenesulfonamide (compound FC86);
N-{(3E)-3-[(1-methyl-1H-imidazol-2-yl)methylidene]-2-oxo-2,3-dihydro-1H-indol-5-yl}-4-methylbenzenesulfonamide (compound FC91);
(Z)-4-methyl-N-(3-((2-methyl-1H-imidazol-5-yl)methylene)-2-oxoindolin-5-yl)benzenesulfonamide (compound SB14);
(E/Z)—N-(3-((1H-pyrazol-4-yl)methylene)-2-oxoindolin-5-yl)-4-methylbenzenesulfonamide (compound SB18);
(Z)—N-(3-((1H-pyrazol-5-yl)methylene)-2-oxoindolin-5-yl)-4-methylbenzenesulfonamide (compound SB17);
(Z)—N-(3-((1H-imidazol-2-yl)methylene)-2-oxoindolin-5-yl)-4-methylbenzenesulfonamide (compound SB19);
N-(3-((2-aminopyridin-3-yl)methylene)-2-oxoindolin-5-yl)-4-methylbenzenesulfonamide (compound SB20);
N-(3-(9H-fluoren-9-ylidene)-2-oxoindolin-5-yl)-4-methylbenzenesulfonamide (compound SB21);
(Z)-4-fluoro-N-(2-oxo-3-(thiophen-2-ylmethylene)indolin-5-yl)benzenesulfonamide (compound IT13);
(Z)-4-nitro-N-(2-oxo-3-(thiophen-2-ylmethylene)indolin-5-yl)benzenesulfonamide (compound IT16); and
(Z)—N-(2-oxo-3-(thiophen-2-ylmethylene)indolin-5-yl)benzenesulfonamide (compound IT15).

8. The method claim 7 wherein the compound is

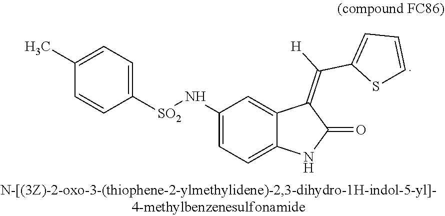 [SMILES: Cc1ccc(S(=O)(=O)Nc2ccc3c(c2)/C(=C/c2cccs2)C(=O)N3)cc1]

9. The method of claim 1, wherein the tumor is a lymphoma, melanoma or anaplastic thyroid cancer ATC.

10. A 2-oxoindole compound of Formula (I) or a pharmaceutically acceptable salt thereof 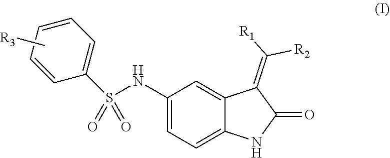
wherein
R₁ and R₂ are, independently from each other, hydrogen, 1H-imidazoly-2-yl, thienyl and 2-methyl-1H-imidazolyl, 2-aminopyridinyl, 1H-pyrazolyl, with the proviso that one of R₁ and R₂ is hydrogen; or R₁ and R₂ together form 9H-fluorene
R₃ is hydrogen, (C₁-C₃)alkyl; halogen or NO₂,
with the proviso that when R₁ or R₂ is thienyl, then R₃ is not methyl.

11. The 2-oxoindole compound of Formula (I) or a pharmaceutically acceptable salt thereof of claim 10, wherein R₁ or R₂ is 1H-pyrazolyl, preferably 1H-pyrazol-2-yl or 1H-pyrazol-5-yl.

12. The 2-oxoindole compound of Formula (I) or a pharmaceutically acceptable salt thereof of claim 10 wherein one of R₁ and R₂ is thienyl.

13. The 2-oxoindole compound of Formula (I) or a pharmaceutically acceptable salt thereof of claim 10, wherein R₃ is methyl.

14. The 2-oxoindole compound of Formula (I) or a pharmaceutically acceptable salt thereof of claim 10, wherein the compound of formula (I) is selected from the group consisting of:

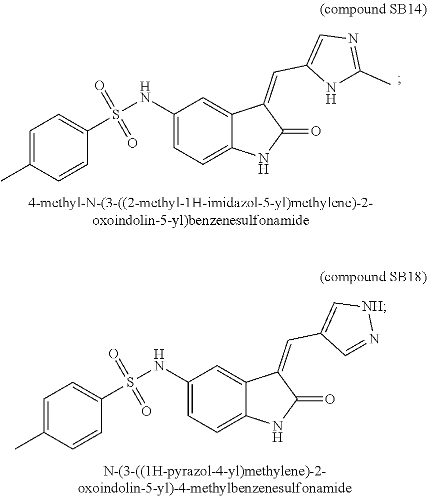

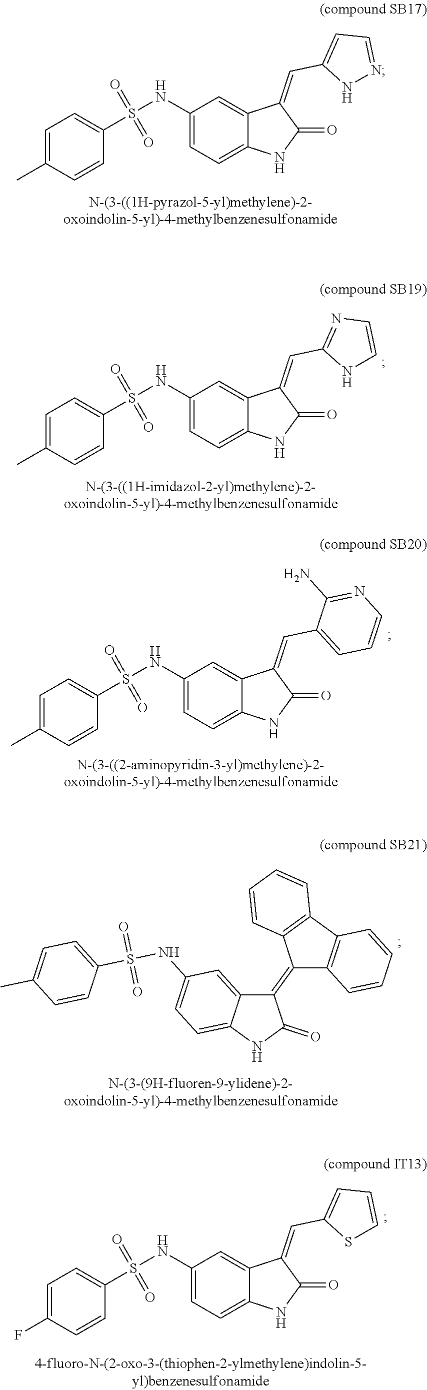 [SMILES: Cc1ccc(S(=O)(=O)Nc2ccc3c(c2)/C(=C/c2ccn[nH]2)C(=O)N3)cc1 ; Cc1ccc(S(=O)(=O)Nc2ccc3c(c2)/C(=C/c2ncc[nH]2)C(=O)N3)cc1 ; Cc1ccc(S(=O)(=O)Nc2ccc3c(c2)/C(=C/c2cccnc2N)C(=O)N3)cc1 ; Cc1ccc(S(=O)(=O)Nc2ccc3c(c2)C(=C2c4ccccc4-c4ccccc42)C(=O)N3)cc1 ; O=C1Nc2ccc(NS(=O)(=O)c3ccc(F)cc3)cc2/C1=C/c1cccs1]

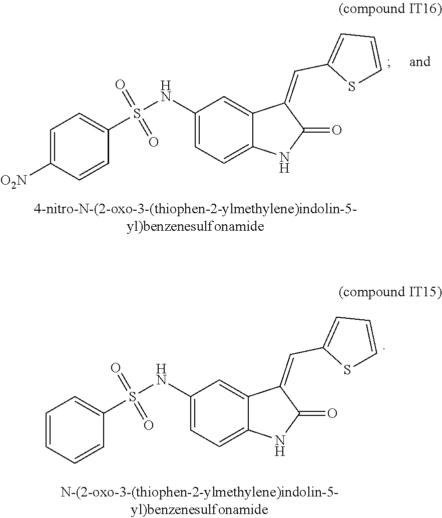 [SMILES: O=C1Nc2ccc(NS(=O)(=O)c3ccc([N+](=O)[O-])cc3)cc2/C1=C/c1cccs1 ; O=C1Nc2ccc(NS(=O)(=O)c3ccccc3)cc2/C1=C/c1cccs1]

15. The 2-oxoindole compound of claim 10 wherein the compound of formula (I) or a pharmaceutically acceptable salt thereof is selected from the group consisting of:
(Z)-4-methyl-N-(3-((2-methyl-1H-imidazol-5-yl)methylene)-2-oxoindolin-5-yl)benzenesulfonamide (compound SB14);
(E/Z)—N-(3-((1H-pyrazol-4-yl)methylene)-2-oxoindolin-5-yl)-4-methylbenzenesulfonamide (compound SB18);
(Z)—N-(3-((1H-pyrazol-5-yl)methylene)-2-oxoindolin-5-yl)-4-methylbenzenesulfonamide (compound SB17);
(Z)—N-(3-((1H-imidazol-2-yl)methylene)-2-oxoindolin-5-yl)-4-methylbenzenesulfonamide (compound SB19);
N-(3-((2-aminopyridin-3-yl)methylene)-2-oxoindolin-5-yl)-4-methylbenzenesulfonamide (compound SB20);
N-(3-(9H-fluoren-9-ylidene)-2-oxoindolin-5-yl)-4-methylbenzenesulfonamide (compound SB21);
(Z)-4-fluoro-N-(2-oxo-3-(thiophen-2-ylmethylene)indolin-5-yl)benzenesulfonamide (compound IT13);
(Z)-4-nitro-N-(2-oxo-3-(thiophen-2-ylmethylene)indolin-5-yl)benzenesulfonamide (compound IT16); and
(Z)—N-(2-oxo-3-(thiophen-2-ylmethylene)indolin-5-yl)benzenesulfonamide (compound IT15).

16. (canceled)

17. A method for the treatment of a tumor comprising the step of administering the 2-oxoindole compound or a pharmaceutically acceptable salt thereof of claim 10, wherein the tumor selected from the group consisting of a lymphoma, chronic lymphocytic leukemia (CLL), Ewing sarcoma, colon cancer, melanoma and anaplastic thyroid cancer (ATC).

18. A pharmaceutical composition comprising the 2-oxoindole compound or a pharmaceutically acceptable salt thereof of claim 10 and a pharmaceutically acceptable carrier.
